(19) United States
(12) Patent Application Publication
(10) Pub. No.: US 2003/0228566 A1
(43) Pub. Date: Dec. 11, 2003

(54) Method of and apparatus for screening for drug candidates

(75) Inventors: Hua Mao, San Diego, CA (US); Lid B. Wong, Elmhurst, IL (US); Tarun Chandra, Gurnee, IL (US); Donovan B. Yeates, Chicago, IL (US)

Correspondence Address: WOOD, PHILLIPS, KATZ, CLARK & MORTIMER, 500 W. MADISON STREET, SUITE 3800, CHICAGO, IL, 60661, US

(73) Assignee: BioTechPlex Corporation

(21) Appl. No.: 10/166,895

(22) Filed: Jun. 11, 2002

(51) Int. Cl. C12Q 1/00

(52) U.S. Cl. 435/4

(57) ABSTRACT

An apparatus is disclosed for screening a compound by monitoring its interactions with a specimen having fluorophore loaded target cells. The apparatus comprises an optical illumination unit comprising a light source wherein light from the light source is directed to illuminate the specimen; a fluorescence discrimination unit which is coupled to receive emitted light from the specimen and separate at least three emitted wavelengths of light from said emitted light; and a fluorescence detection unit which is coupled to the fluorescence discrimination unit counts photons emitted by the wavelengths of emitted light. A method of screening a compound by monitoring its interactions with a specimen having fluorophore loaded target cells is also described. The method comprises the steps of coupling a light source to the specimen to illuminate the specimen; separating at least three wavelengths of light emitted by the specimen, and detecting photon counts from the three emitted wavelengths of light.

### GOVERNMENT SUPPORT

The present invention was made with U.S. Government support from the National Institutes of Health, National Heart, Lung, and Blood Institute, under grant No. HL 59816 and from the State of Illinois Technology Challenge Grant Program. The U.S. government and the State of Illinois have certain rights in this invention.

### FIELD OF THE INVENTION

This invention relates generally to diagnostic systems, and in particular, to a method of and apparatus for screening for drug candidates.

### BACKGROUND OF THE INVENTION

The continued and improved health of the pharmaceutical industry and the nation is dependent on a constant supply of new lead compounds that will result in new therapeutic treatments for disease. This requires the screening of a library of candidate compounds for specific biological activity that will result in efficacious treatment with minimal side effects and low toxicity. Ion channels are central to many physiological processes and have been implicated in several diseases, e.g. cystic fibrosis and hypertension. With the explosive growth in knowledge related to the human genome, ion channels have become an increasingly important target class for new drug development. Existing cell-based high-throughput screening assays provide the measurable physiological outputs that can be linked to ion channel function but fall short when trying to meet the competing demands of high-throughput and the millisecond time scale temporal resolution requirements of ion channel responses.

More importantly, these existing high-throughput screening devices generally do not provide detailed mechanistic information on the potential drug candidates classified as “hits.” These potential drug candidates then undergo a low throughput, high content screening in order to become a “lead compound.” Subsequently, specific assays are developed to establish the mechanisms of the signal transduction pathways to verify that the lead compound is worthy of follow-up study. Such a set of procedures is extremely expensive and time-consuming, especially considering that the vast majority of compounds undergoing such screening do not become drugs.

Clearly, any new technique developed to improve the efficiency of this time-consuming and cost-intensive drug discovery process will be highly beneficial. One such approach to reduce the number of stages of drug discovery involves cellular assay screens. Cellular assay techniques often use fluorescence detection, which has major advantages as compared to other investigation methods. These include high sensitivity, wide dynamic range, and capability of remote detection of the signals from the samples. Fluorescence detection techniques enable monitoring of rapid dynamic changes of the concentration of substances of interest in living cells and biological tissues. Fluorescence-based measurements have been widely adopted to investigate the signal transduction pathways activated via drug and cell receptor, ion channel, or other cell-specific interactions.

None of the cell-based assay technologies uses multiple simultaneous measurements. There are a number of fluorescence detection devices available for detecting intracellular constituents of interest in biological samples. Most of these devices use epi-illumination fluorescence microscopy and can only perform one fluorophore measurement at a time. In such systems, an excitation wavelength is chosen by filtering a broad band light source that is transmitted through a microscope objective to illuminate the specimen. Light emitted from the specimen is collected by the same microscope objective, filtered and detected by either a charge coupled-device (CCD) camera or a photomultiplier tube (PMT).

Single fluorophore detection approaches have the limitation that they can only detect one event at a time. For example, Fura-2 fluorophore detection has been widely used to measure intracellular calcium ion concentration as a second messenger to indicate whether or not a G protein coupled receptor has been activated by a drug. However, the actual physiological situation is more complex. In some cases, a single receptor can activate different G proteins and thereby induce dual or multiple signaling routes which lead to the production of multiple second messengers. In other cases, multiple receptors can converge on a single G protein that has the capability of integrating different signals. Different signaling pathways also interact with each other to carry out complex cellular events or permit fine-tuning of cellular activities required in developmental and physiological processes. In this regard, a single ligand may initiate more than one effector protein and thereby initiate a complex signaling network. Single fluorophore systems cannot detect such interactions among ionic and signal transduction pathways.

The use of more than one fluorophore as a way of increasing the sensitivity and precision of assays has been recognized. Two fluorophores have been used in a cross-correlation method to determine the kinetics of enzyme cleavage of a molecule to which the two fluorophores were attached to different parts of the cleaved molecule. However, unlike the present invention, this technique has been used to assay a single event and cannot be used to assay a complex of events characteristic of a living cell.

The detection of several cellular events simultaneously would greatly increase the volume and quality of information available from each screening assay. The need for such a multiple assay has been widely recognized. Previous approaches involved analyzing concentration measurements of cellular constituents produced in response to various concentrations of drug candidates, but unlike the present invention do not provide kinetic information.

A multi-fluorophore detection system that can be used to detect multiple cellular kinetic events simultaneously is very important for the delineation and understanding of ionic and signal transduction pathways and their interactions, multiple signal transduction pathways activation, and the corresponding down-stream cascades initiated by a single ligand. The availability of such a multi-fluorophore system has the potential to greatly improve the cost-effectiveness of drug discovery and to compress the drug discovery process timeline. For example, considering the regulation of epithelial cell function, epithelial cell functions are temporally regulated by sequential activation of multiple major ionic channels and transporters that regulate intracellular Ca⁺⁺, Na⁺, K⁺, and Cl⁻, which in turn modulate the cell membrane potential. Individual measurements of intracellular Na⁺([Na⁺]i), Ca⁺⁺ ([Ca⁺⁺]i), Cl⁻ ([Cl⁻]i) concentrations and cell membrane potential suggest that they are central to many fundamental physiological and pathophysiological mechanisms. The specificity and sensitivity of [Ca⁺⁺]i, [Na⁺]i, [Cl⁻]i and cell membrane potential are linked to many of these mechanisms. Thus, direct measurement of [Ca⁺⁺]i, [Na⁺]i, [Cl⁻]i and cell membrane potential are appropriate end point indicators to evaluate drug candidates for potential therapeutic intervention. The site and mechanism of action of a test compound can be identified with a high degree of specificity by simultaneously characterizing agent-induced dynamic (and spatial) responses of the fluorescence from multiple fluorophores. Each of these fluorophores is sensitive to an agent-induced change in molecular state or concentration of a specific ion or lipid membrane potential. Since each of these parameters is dependent on specific cellular mechanisms that may or may not be coupled, the resultant combinatory data set can give unique characteristic information on the drug candidate used to challenge the cells. Such data can not be reliably derived from the measurement of a single fluorophore or from sequential measurements of the response of each of several fluorophores.

Accordingly, there is a need for an improved method of and apparatus for screening a drug candidate.

### BRIEF DESCRIPTION OF THE DRAWINGS

FIG. 1 is a block diagram of a system for screening for drug candidates according to the present invention;

FIG. 2 is a more detailed block diagram of a system for screening for drug candidates according to a particular alternate embodiment of the present invention; and

FIG. 3 is a flow chart for a method of screening for drug candidates according to the present invention.

FIG. 4 is a more detailed flow chart for a method of screening for drug candidates according to a particular alternate embodiment of the present invention.

FIG. 5 is a flow chart for a method of screening for drug candidates according to an alternate embodiment of the present invention.

FIG. 6 shows spectral characteristics of dichroic mirrors for simultaneous measurement of a fluorescein-based fluorophore, a dihydroquinoline-based fluorophore, and a styryl-based fluorophore according to the present invention.

FIG. 7 is an example of the cumulative dose kinetic responses of Ca⁺⁺, Cl⁻ and cell membrane potential to incrementally increasing concentrations of Glibenclamide in normal human bronchial epithelial cells according to the present invention.

FIG. 8 is an example of the cumulative dose kinetic responses of Ca⁺⁺, Cl⁻ and cell membrane potential in NHBE cells to incrementally increasing concentrations (0.01 □M to 1 mM) of uridine triphosphate (UTP) according to the present invention.

FIG. 9 is an example of the cumulative dose kinetic responses of Ca⁺⁺, Cl⁻ and cell membrane potential in NHBE cells to incrementally increasing concentrations (0.01 mM to 1.0 mM) of 1,3-dihydro-1-[2-hydroxy-5-(trifluoromethyl)phenyl]-5(trifluoromethyl)-2H-benzimidazol-2-one (NS 1619) according to the present invention.

The accompanying drawings, which are incorporated in and form a part of this specification, illustrate embodiments of the invention and, together with descriptions, serve to explain the principles of the invention. They are not intended to limit the scope of the invention to the embodiments described. It will be appreciated that various changes and modifications can be made without departing from the spirit and scope of the invention as defined in the appended claims.

### SUMMARY OF THE INVENTION

The present disclosure relates to a multi-signal cell-based drug screening system utilizing the simultaneous measurement of the time-dependent fluorescence from three or more fluorophores activated by a drug candidate. The system advances the current state of the art by providing higher sensitivity and specificity than present systems. With improved light collection and reduced background noise, signal/noise ratio is also greatly improved. The use of dichroic polarizer-analyzers greatly diminishes interference from incident light. The kinetics of the cellular events can be measured for the first time on a millisecond time scale through the use of high bandwidth, high frequency photon counting. By the simultaneous measurement of several fluorescent signals, complex cellular responses to drug candidates can be elucidated. Thus, detailed characterization of target cells and their response to drug candidates become possible for high throughput drug screening.

The system consists of an optical excitation system containing light sources that emit at least two pre-determined wavelengths of light together with at least two dichroic mirrors or equivalent filters to direct the incident light to the specimen; a specimen holding/indexing system preferably comprising an inverted fluorescence microscope or an optical scanner; a fluorescence separation system comprising of at least two long-pass dichroic mirrors or equivalent filters to direct and separate at least three emitted wavelengths and direct them to the photo-detectors; a fluorescence photodetection system comprising a plurality of dichroic polarizer-analyzers, a plurality of interference filters for the respective emission wavelengths, and a plurality of photon detectors; and a multi-channel transistor-transistor logic (TTL) counter and interfaced computer control system that processes and displays a minimum of 3 fluorescence signals in real-time at a bandwidth of 1 MHz each. The fluorophores target a major cation, a major anion, and the cell membrane potential. For example, the major cation could be Na⁺, K⁺, or Ca⁺⁺, while the major anion could be Cl⁻, or HCO₃⁻. The three detectors of the present invention could be designed to detect a major cation, a major anion, and the cell membrane potential, respectively.

The system of the present invention provides a fluorescence detection system that has a high signal to background noise ratio; high sensitivity simultaneous detection of three fluorescence emissions and their kinetics from such biological specimens as cells, tissues, organs and proteins; a high speed, real-time detection system that captures cellular events occurring on a millisecond time scale and, thus, allows for the first time, detailed temporal characterization of cellular responses to drug candidates.

By using the cell-based fluorescence detection invention disclosed herein, both non-specific target activated and specific physiological activity and toxicity can be determined at the cellular level in a manner that is not possible when screening at the molecular or enzymatic level. An additional use of the method and apparatus of the present invention is to provide a cellular screen for validation of “hits” from such molecular or enzymatic screens. Changes in fluorescence kinetics for cellular fluorophore reportable molecular species in time intervals on the order of milliseconds can be detected.

### DESCRIPTION OF THE PREFERRED EMBODIMENTS

Reference will now be made in detail to the preferred embodiments of the invention, examples of which are illustrated in the accompanying drawings. While the invention will be described in conjunction with the preferred embodiments, it will be understood that they are not intended to limit the invention to these embodiments. The invention is intended to cover alternatives, modifications and equivalents, which may be included within the invention as defined by the appended claims.

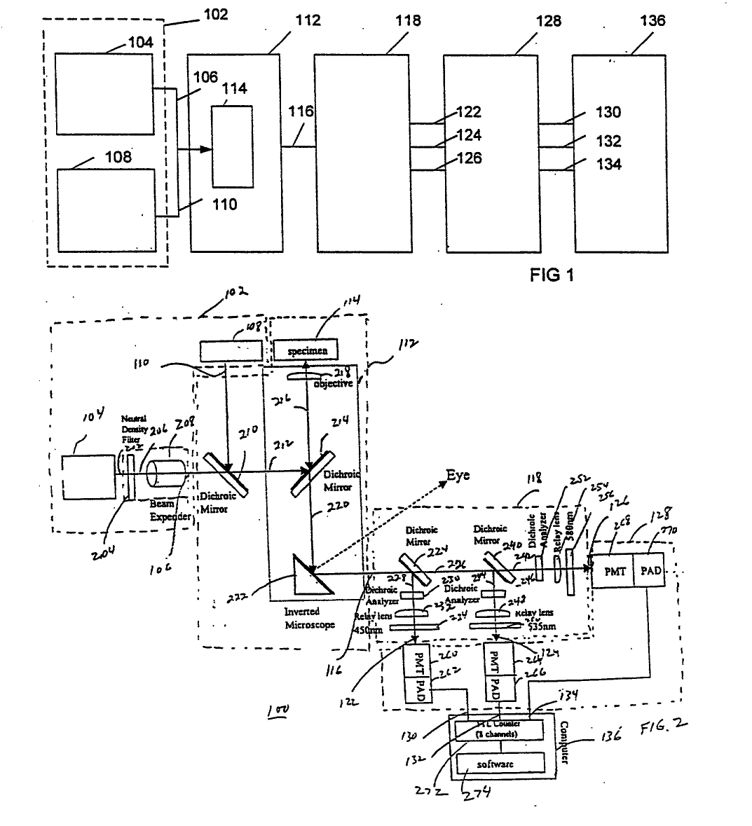

Turning now to FIG. 1, a block diagram of a drug screening system 100 is shown. In particular, a light source block 102 comprises a first light source 104 generating a first light beam 106, and a second light source 108 generating a second light beam 110. The light source block 102 preferably includes at least 2 predetermined excitation wavelengths of polarized light. In the preferred embodiment, the light source block 102 consists of a light source assembly containing a low power (<50 mW) polarized argon laser merged with a xenon light source. A microscope 112 holds and indexes one or more fluorophore-loaded specimens 114. The specimens 114 are maintained by the microscope 112 which receives the light beams 106 and 110. Light beam 116 emitted by the specimen is coupled to a fluorescence separation device 118. The fluorescence separation device 118 generates a plurality of wavelengths of light 122, 124, and 126. Although three wavelengths of light are shown here, any number of wavelengths could be generated. The wavelengths of light 122, 124 and 126 are coupled to a photon detector block 128 having a plurality of photon detectors. The photon detectors detect and count photon emissions from the wavelengths of light 122, 124 and 126, and couple the counts 130, 132 and 134 to a computer 136. A response profile of the target cells is generated based upon the photon emission counts.

Turning now to FIG. 2, a more detailed block diagram of the drug screening system 100 is shown. In particular, the light source 102 comprises the first light source 104 which generates a laser beam 202. The laser beam 202 is coupled to a filter 204. The filter could be, for example, a neutral density filter which will reduce the intensity of the laser beam in order to reduce any damage to the specimen from the laser beam. In particular, if the intensity of the laser beam is too bright, the dye in the specimen will bleach. The filtered laser beam 206 which is output from the filter 204 is coupled to a beam expander 208. The beam expander 208 widens the laser beam, and generates the first light beam 106.

In one embodiment, the first light source 104 comprises a polarized argon ion laser used as an excitation source. The laser beam 202 passes through the filter 204 which could be, for example, a neutral density filter, to the beam expander 208 which could be, for example, a 10× beam expander. The light beam which is output from the beam expander 208 in the embodiment of FIG. 2 is coupled to a dichroic mirror 210. In particular, in addition to passing the first light beam 106, the dichroic mirror deflects the second light beam 110. The dichroic mirror 210 could be, for example, a 45° long pass dichroic mirror which passes the wavelength of the first light beam 106 and reflects the other wavelengths. FIG. 6 shows spectral characteristics of the dichroic mirrors which could be used for simultaneous measurement of a fluorescein-based fluorophore, a dihydroquinoline-based fluorophore and a styryl-based fluorophore according to the present invention. Although the dichroic mirrors 210 and 214 are shown as a part of the microscope 112, the dichroic mirrors could be separate from or attached to a conventional microscope.

The second light beam 110 could be a monochromatic light beam generated from a xenon lamp and used as an excitation light source which is directed to and deflected by the dichroic mirror 210. Alternatively, monochromatic light from sources such as a mercury arc lamp might also be used. The second dichroic mirror 214 is positioned to deflect the combined light beam 212 to create an incident light beam 216 which is coupled to an objective lens 218 prior to hitting the specimen 114. The merged light beams, reflected 90° perpendicularly by a 45° band pass dichroic mirror mounted beneath the objective of the microscope, are focused onto the specimen by the objective lens 218.

The dichroic mirror 214 also passes light emitted by the specimen 114. A passed light beam 220 is provided to an 80% Thompson reflective prism 222 contained within the inverted microscope 112. The prism deflects the light beam to generate the deflected light beam 116. Fluorescent wavelengths emitted from the specimen 114 pass and are preferably reflected by the 80% Thompson reflective prism inside the microscope to the side port of the microscope. The inverted microscope also enables a viewer to view the reflected light beam 220 to ensure that the incident light beam 216 is properly focused on the specimen. The number of fluorescence wavelengths depends upon the number of fluorophores in the specimen. The embodiment of FIG. 2 is designed to detect three fluorescent wavelengths, although it could be designed to detect any number of wavelengths.

Typically, issues in fluorescence detection include the reduction of background noise in the detection system, excitation source associated optics (dichroic mirror, interference filters, focusing lenses, etc.), the substrate containing the sample to be analyzed, and the emission filters in the multiple fluorophore detection system. When multiple wavelengths of source light and multiple wavelengths of emission are involved, reduction of background signal becomes more critical. The key challenge for multiple fluorophore detection in the epifluorescence mode is to effectively separate and collect photons from multiple emission wavelengths with minimal photon loss, and without generating a high background signal from multiple wavelengths of the incident light source.

An emitted fluorescence light beam consisting of the three wavelengths is preferably directed to another long pass dichroic mirror which reflects the shortest wavelength and allows the passage of the other two longer wavelength fluorescent signals. The fluorescence wavelength reflected by the long pass dichroic mirror preferably passes through a dichroic polarizer-analyzer, an interference filter for the wavelength, and is focused by a relay lens onto a photon counting photomultiplier tube (PMT). Preferably, the fluorescence separation device 118 directs each component wavelength of emission fluorescence to each individual photon detector, and at the same time reduces the reflection noise from the excitation light source. The use of dichroic polarizer-analyzers in the detection path greatly reduces interference from the incident wavelengths and increases the signal to noise ratio. To select the preferred dichroic polarizer-analyzer for a specific application, it is necessary to determine the signal to noise ratio or signal to background level for a particular emission wavelength when a polarized excitation source is used. Signal to noise ratios can be determined by comparing the magnitude of the light emissions from a defined amount of fluorescent material measured to the noise obtained by measuring an empty addressable well under identical conditions. “Addressable well” refers to a spatially distinct location on one well of a multi-well chamber, which has a thin bottom (˜0.17 mm #1 cover glass) within the microscope objective focal length or within the range of another detection device that serves the same purpose, such as an optical scanner, and with open access at the top.

Referring particularly to the embodiment of FIG. 2, the reflected light 116 is coupled to a third dichroic mirror 224 which separates the reflected light 116 into a passed light beam 226 and a deflected light beam 228. The deflected light beam 228 is preferably of a first wavelength. The deflected light beam 228 is coupled to a dichroic polarizer-analyzer 230, followed by a relay lens 232 and a filter 234. The passed light 226 is provided to a fourth dichroic mirror 240 which also passes a portion of the light to generate a passed light beam 242 and a deflected light beam 244 of a second wavelength. The deflected light beam 244 is provided to another dichroic polarizer-analyzer 246, a relay lens 248 and a filter 250. Finally, the passed light beam 242 of a third wavelength is coupled to a dichroic polarizer-analyzer 252, a relay lens 254 and filter 256. The relay lens focuses the deflected light beams to their respective counters, while the filters, preferably band pass filters, pass the desired frequency of the deflected light beam. Accordingly, the fluorescence separation device 118 generates light beams from the specimens having three different wavelengths.

Each of the three light beams, after being filtered by the dichroic polarizer-analyzers, relay lenses, and filters, is provided to a PMT and a pulse amplifier and discriminator (PAD). The output of each PMT and PAD is coupled to a computer 136 comprising a TTL counter 272 and associated software 274. The resulting current pulses generated by the PMT 260, 264, and 268 are converted to 5V transistor-transistor logic (TTL) pulses by the PADs 262, 266 and 270. The resulting TTL pulses 130, 132, and 134 from each of the PADs 262, 266, and 270, respectively, are preferably coupled to TTL counter 272, which could be for example, a 5-channel, 5 MHz TTL counter interfaced to a computer. The data are processed by software 274 and the results could be displayed on a screen in real-time.

The fluorescence detection system 118 and 128 preferably includes at least three photon sensitive detectors, such as photomultiplier tubes (PMTs), charge coupled devices (CCDs), or photodiodes. In the preferred embodiment, such PMTs have a maximum count rate (random pulse) up to 3×10⁷ cps for simultaneous photon detection and quantification of at least three emission wavelengths. Such PMTs usually exhibit good linearity up to 10⁷ cps. The detectors preferably function in the epifluorescence mode where the preferred illumination is from the bottom of the addressable well and the preferred collection of the emitted light signal is also from the bottom of the addressable well.

Preferably, a multi-channel TTL counter interfaced to a computer control system that processes and displays a minimum of three fluorescence signals in real time, each with a minimum of 1 MHz bandwidth, should be used. Preferably, the data processing and control unit converts current pulses generated from a PMT to 5V TTL pulses that are further counted by the multi-channel TTL counter 130 interfaced to a computer. Photon counts from multiple detectors are measured intermittently. Counts from each of the emitted multiple wavelengths are preferably displayed simultaneously on a computer screen in real time.

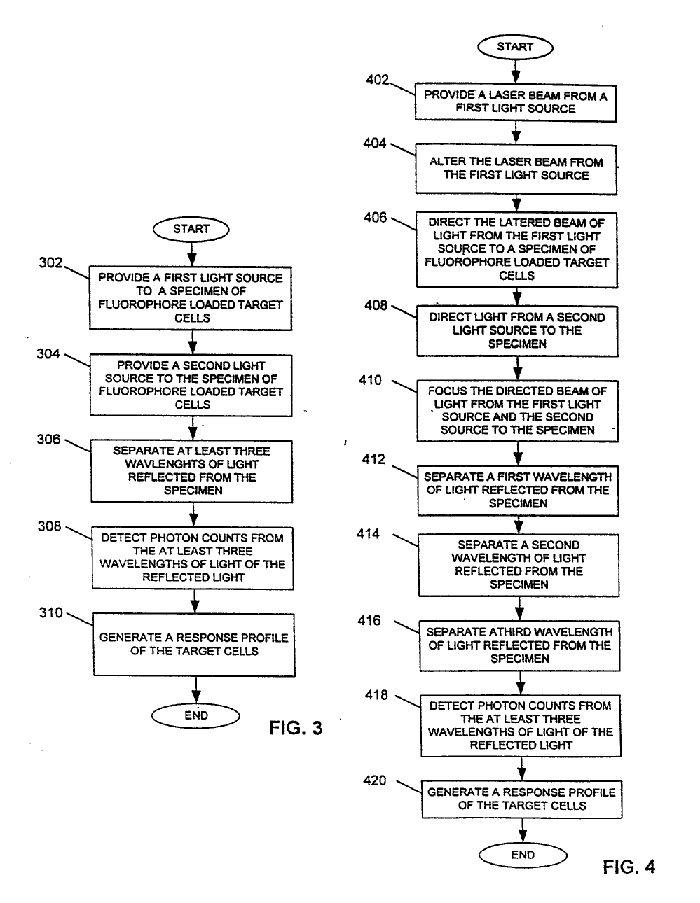

Turning now to FIG. 3, a flow chart shows a method of screening for drug candidates according to the present invention. A first light source is provided to a specimen of fluorophore loaded target cells at a step 302. A second light source is also provided to the specimen of fluorophore loaded target cells at a step 304. At least three wavelengths of light emitted by the specimen are separated at a step 306. Photon counts from at least three wavelengths of light are detected at a step 308. A response profile of the target cells is then generated at a step 310.

Turning now to FIG. 4, a flow chart shows a more detailed method of screening for drug candidates according to the present invention. In particular, a laser beam from a first light source is provided at a step 402. The laser beam from the first light source is altered to generate an appropriate light beam at a step 404. The altered beam of light from the first light source is directed to a specimen of fluorophore loaded target cells at a step 406. Light from a second light source is directed to the specimen at a step 408. The directed beam of light from the first light source and the second light source is focused on the specimen at a step 410. A first wavelength of light from light emitted by the specimen is separated at a step 412. A second wavelength of light from light emitted by the specimen is separated at a step 414. Finally, a third wavelength of light from light emitted by the specimen is separated at a step 416. Photon counts from the three wavelengths of light are detected at a step 418. A response profile of the target cells is generated based upon the photon count at a step 420. It should be understood that the methods of FIGS. 3 and 4 could be performed by the system of screening for drug candidates of FIG. 2, or some other suitable device.

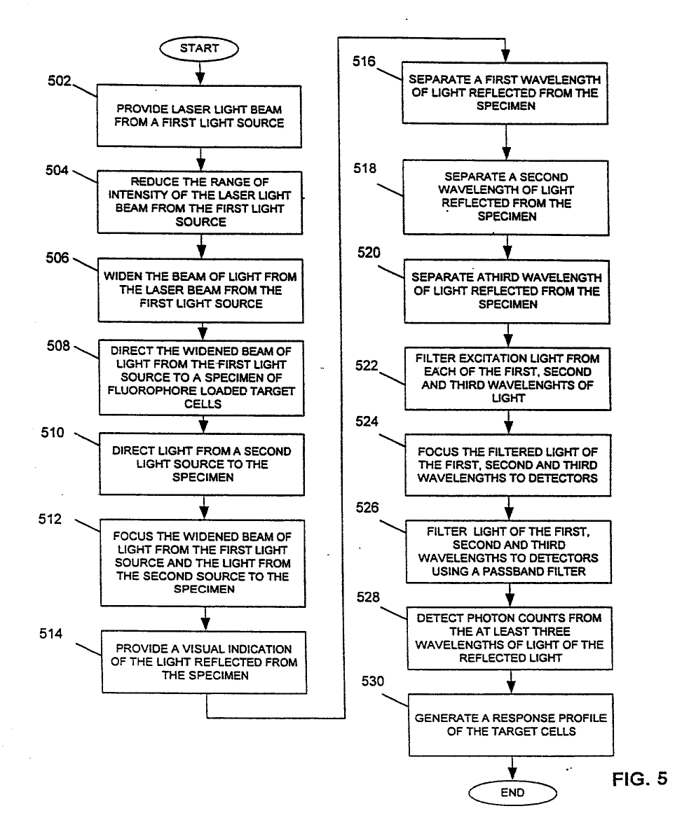

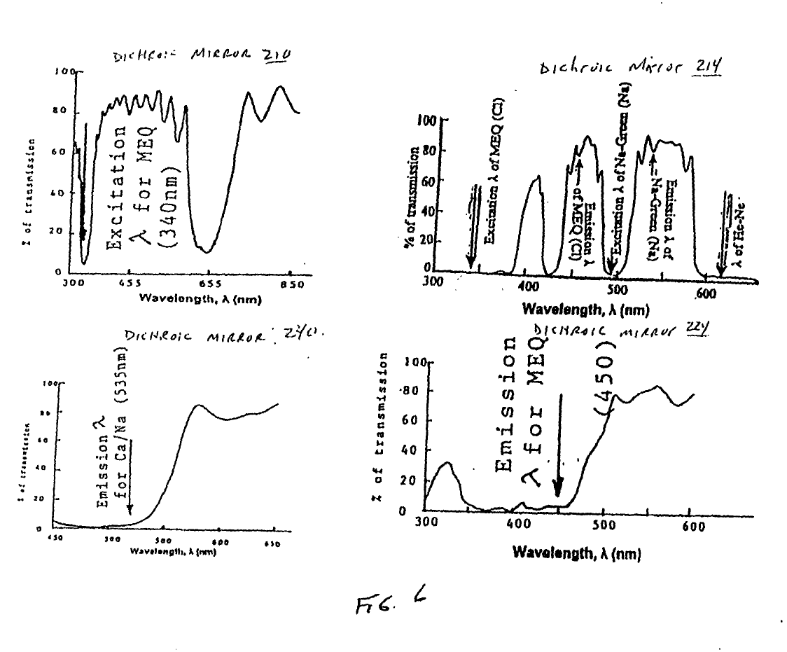

Turning now to FIG. 5, a flow chart shows another method of screening for drug candidates according to the present invention. A laser light beam from a first light source is provided at a step 502. The range of intensity of the laser light beam from the first light source is reduced at a step 504. The range of intensity could be reduced, for example, by a neutral density filter, such as the filter 204 of FIG. 2. The beam of light from the laser beam from the first light source is widened at a step 506. The beam could be widened, for example, by a beam expander, such as the beam expander 208 of FIG. 2. The widened beam of light from the first light source is directed toward a specimen of fluorophore loaded target cells at a step 508. Light from a second light source is directed to the specimen at a step 510. The widened beam of light from the first light source and light from the second light source are focused on the specimen at a step 512. The beams of light could be focused on a specimen by using a lens, such as the objective lens 218 of FIG. 2. A visual indication of light emitted by the specimen is preferably provided at a step 514. The visual indication could be provided by an inverted microscope, such as the inverted microscope 112 of FIG. 2. The visual indication enables an operator who is screening drugs to ensure that the beams of light directed on a specimen are properly focused on the specimen.

A first wavelength of light emitted by the specimen is separated at a step 516. The first wavelength of light could be separated, for example, by a dichroic mirror, such as dichroic mirror 224 of FIG. 2. Similarly, a second wavelength of light emitted by the specimen is separated at a step 518. The second wave length of light could be separated by a second dichroic mirror, such as dichroic mirror 240 of FIG. 2. Finally, a third wavelength of light emitted by the specimen is separated at a step 520. The third wavelength of light could be, for example, the light passed by the dichroic mirrors 224 and 240 of FIG. 2. Excitation light is then filtered from each of the first, second and third wavelengths of light at a step 522. For example, dichroic analyzers, such as dichroic analyzers 230, 246 and 252 of FIG. 2 could be used to filter excitation light. The filtered light of the first, second, and third wavelengths is focused to detectors at a step 524. For example, relay lenses 232, 248, and 254 of FIG. 2 could be used to focus the wavelengths of light. Each of the three wavelengths of light are then passed through a separate interference filter at a step 526. For example, filters 234, 250 and 256 of FIG. 2 could be selected to pass the three wavelengths of light, respectively. Finally, photon counts from each of the three wavelengths of light are detected at a step 528 and a response profile of the target cells is generated at a step 530.

The following examples use the experimental protocols described below unless specified otherwise. They are intended for purposes of illustration only and should not be construed to limit the scope of the invention as defined in the claims appended hereto.

Normal human bronchial/tracheal epithelial cells (NHBE, Clonetics) were cultured in T-25 cm² flasks at 37° C., 5% CO₂ using Bronchial/Tracheal Epithelial Cell Growth Medium containing Retinoic Acid (BEGM, w/RA, Clonetics). When the NHBE cells in the T-25 flasks reached 60%-80% confluency, the cells were passaged using a seeding density of 3500 cells/cm². A portion of the cells was passaged in T-25 flasks again while the remaining cells were seeded on UV-exposed Vitrogen (pH balanced 1:1 BEGM to Vitrogen) coated 4-well cover glass chambers (LabTek II). NHBE cells normally attached to the collagen-coated cover glass chamber within 24 hrs. Prior to these cells reaching 60% confluency, they were used for all the experiments described below. Cells used in all the experiments were either 2^{nd} or 3^{rd} passage cells maintained in Bronchial/Tracheal Epithelial Cell Growth Medium with Retinoic Acid (BEGM, w/RA, Clonetics).

The following procedures for loading the cells with fluorophore were employed. Balanced Hank's (BH) solution without phenol red was used as the medium for all the fluorophore preparations and cell washings unless stated otherwise. Fluo-3 (a Ca⁺⁺ indicator), di-MEQ (a Cl⁻ indicator) and RH421 (a cell membrane potential indicator) were loaded into cells sequentially at room temperature. Cells were first incubated with 8 μM Fluo-3 solution for 60 minutes, followed by incubating with 50 μM di-MEQ for 5 minutes and then finally with 10 μM RH421 for 5 minutes. Extraneous dyes were washed with BH solution between each fluorophore loading procedure. The cells were allowed to stabilize in BEGM for a minimum of 15 minutes at room temperature prior to the beginning of each experiment.

Experiments were performed at room temperature. All tested agents were prepared in Balanced Hank's Solution. One of the wells of the cover glass chamber was placed on the stage of the inverted microscope and the cells loaded with the fluorophores were visually focused with the violet, green and orange emitted light from the cells approximately in the same focal plane. At a sampling frequency of 100 Hz, cells with the following photon counts were chosen for the study: 20 to 50 counts per channel (cpc) for Ca⁺⁺ fluorescence, 70 to 200 cpc for Cl⁻ fluorescence, and 20 to 50 cpc for cell membrane potential fluorescence. After establishing a 2 minute baseline, increasing doses of the agent of interest were added topically to the wells 2 minutes apart. At the end of each experiment, a toxic dose of the agent of interest was added to the sample to either shrink or swell the cells beyond their normal cell volume regulatory range. This caused the fluorescence signals of each of these fluorophores to reach either maximum or minimum values. If either one of these fluorescence signals did not reach a maximum or minimum, the experiment was discarded. Background fluorescence was recorded using a cell free area of the same well. If the signal to background ratio was not higher than a factor of 10, the experiments were also discarded.

### EXAMPLE 1

### Cumulative Dose Kinetic Responses of Intracellular Ca⁺⁺, Cl⁻ and Cell Membrane Potential to Glibenclamide.

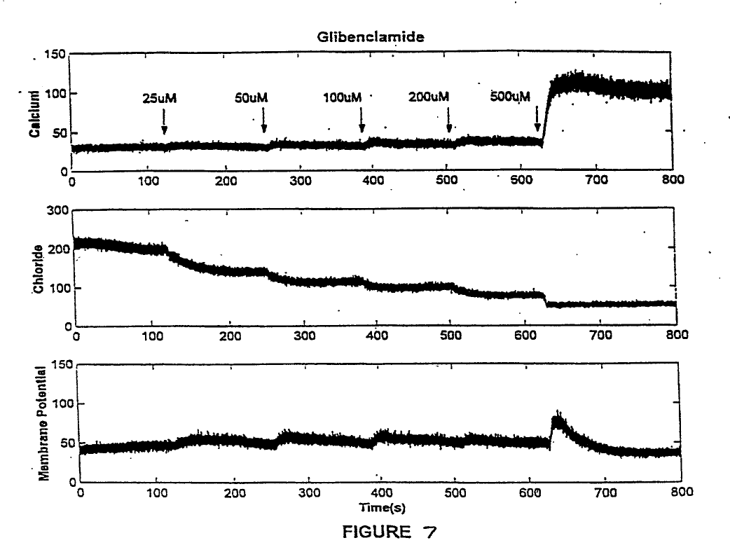

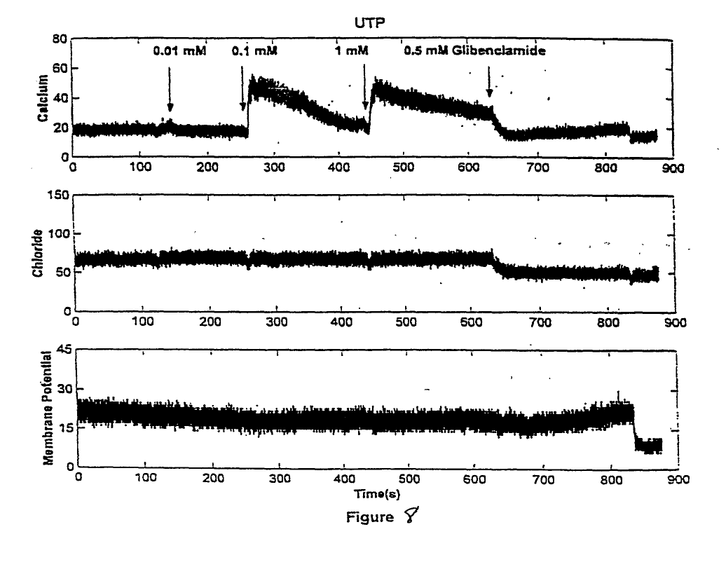

Glibenclamide, a chloride channel blocker in airway epithelial cells predictably increased [Cl⁻]i (the fluorescence of MEQ is inversely proportional to [Cl⁻]i) that in turn hyperpolarized the cell membrane. The responses are shown in FIG. 7. In particular, FIG. 7 shows an example of the cumulative dose kinetic responses of Ca⁺⁺, Cl⁻ and cell membrane potential in normal human bronchial epithelial cells (NHBE) to incrementally increasing concentrations (25 μM to 500 μM) of Glibenclamide, a chloride channel blocker. The kinetic responses were measured as photon counts acquired in 10 ms intervals over >800 seconds. It may be noted that, in normal human epithelial bronchial cells for Glibenclamide concentrations below 500 μM, intracellular Ca⁺⁺ and membrane potential are little affected, while intracellular Cl⁻ declines as Glibenclamide concentration increases. When Glibenclamide concentration reaches 500 μM, however, there is a rapid increase in intracellular Ca⁺⁺, a simultaneous drop in intracellular Cl⁻, and a simultaneous increase in membrane potential. The correlation of these events and their kinetics, observations that can only be made with the present invention, provides unique insights into the mechanism by which Glibenclamide affects the cells.

### Example 2

### Cumulative Dose Kinetic Responses of Intracellular Ca⁺⁺, Cl⁻ and Cell Membrane Potential to Uridine Triphosphate.

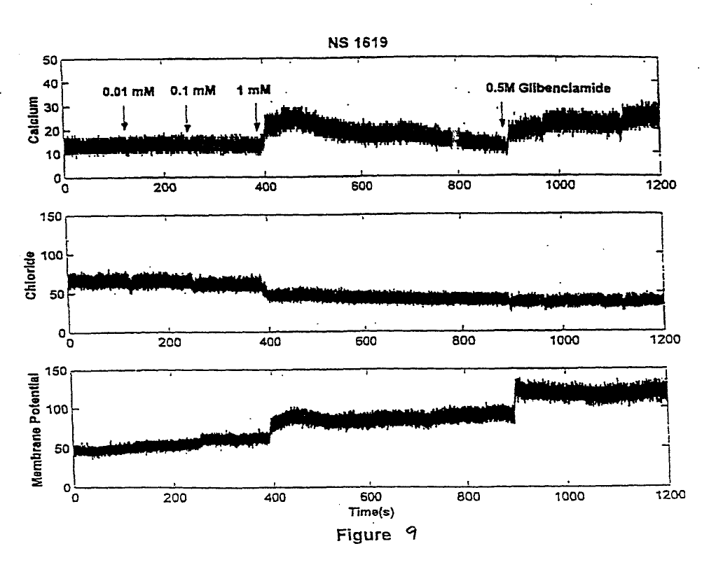

FIG. 8 shows an example of the cumulative dose kinetic responses of Ca⁺⁺, Cl⁻ and cell membrane potential in NHBE cells to incrementally increasing concentrations (0.01 mM to 1 mM) of uridine triphosphate (UTP), a calcium dependent chloride channel activator. UTP is a ligand to the p2Y receptor. The kinetic responses were measured as photon counts acquired in 10 ms intervals over >800 seconds.

Uridine triphosphate (JTP) is a calcium dependent chloride channel activator. The responses to increasing concentrations of UTP are shown in FIG. 8. It may be noted that for UTP concentrations of 0.1 mM, and 1 mM, there is a rapid increase in intracellular Ca⁺⁺, followed by a measurable rate of decline, but essentially no changes in either intracellular Cl⁻ or membrane potential. With the present invention, the kinetics of intracellular Ca⁺⁺ flux out of the cell can be determined, and its relationship to other cellular events can be examined.

### EXAMPLE 3

### Cumulative Dose Kinetic Responses of Intracellular Ca⁺⁺, Cl⁻ and Cell Membrane Potential to 1,3-dihydro-1-[2-hydroxy-5-(trifluoromethyl)phenyl]-5-(trifluoromethyl)-2H-benzimidazol-2-one (NS 1619), a Calcium Sensitive Bk Potassium Channel Activator

Hyperpolarization of the cell membrane can also be induced via different cellular mechanisms such as by decreasing intracellular potassium. FIG. 9 shows an example of the cumulative dose kinetic responses of Ca⁺⁺, Cl⁻ and cell membrane potential in NHBE cells to incrementally increasing concentrations (0.01 mM to 1.0 mM) of 1,3-dihydro-1-[2-hydroxy-5-(trifluoromethyl)phenyl]-5-(trifluoromethyl)-2H-benzimidazol-2-one (NS 1619), a calcium sensitive Bk potassium channel activator. The kinetic responses were measured as photon counts acquired in 10 ms intervals over >900 seconds. It should be noted that the relative potencies of these agents in terms of the mechanisms for eliciting the temporal responses, the duration of the agent actions and the magnitude of hyperpolarization of the cell membrane can be compared and monitored for the first time. The responses to increasing concentrations of 1,3-dihydro-1-[2-hydroxy-5-(trifluoromethyl)phenyl]-5-(trifluoromethyl)-2H-benzimidazol-2-one (NS 1619) are shown in FIG. 9. At 0.01 mM, there is essentially no change in intracellular Ca⁺⁺, nor in intracellular Cl⁻, but a small increase in membrane potential. At 0.1 mM, both intracellular Ca⁺⁺ and Cl⁻ are essentially unchanged, but membrane potential shows a small increase. At 1 mM, intracellular Ca⁺⁺ shows a sharp increase, intracellular Cl⁻ shows a simultaneous sharp decrease, and membrane potential shows a simultaneous sharp increase. The correlation of these events can provide important insights into the underlying mechanisms of the activation. These sets of at least three simultaneous responses to a stimulant can then be used to determine characteristic parameters which together uniquely define the ensemble kinetic response profile of the target cells in the specimen to the stimulant.

The method and apparatus of the present invention find particular application in the delineation of cellular signal transduction pathways and the identification of bioactive agents that activate or modulate these pathways. This technology can be used to improve the efficiency of screening candidates for new drugs. The method and apparatus can be used to combine high throughput screening of drug candidates with high information content. Current technology uses two separate steps, first a rapid initial low-information content step as an initial screen, followed by a second high-information content screen of the drug candidates that survive the first step. The ability to follow three or more cellular signals simultaneously in real time in a single step opens the possibility of learning more about the interaction of complex cellular events than is possible with current technologies. The method and apparatus of the present invention provide a new tool to developing such an understanding. The method and apparatus could also be adapted to simultaneously detect and follow several ion and/or other specie concentrations in body fluids in real time with a time scale resolution of milliseconds. Finally, it should be understood that the control and analysis software developed for this method and apparatus could be applied to other technologies that involve following three or more simultaneous signals in real time with a millisecond or greater time scale resolution.

It can therefore be appreciated that a new and novel method and apparatus for screening a drug has been described. It will be appreciated by those skilled in the art that, given the teaching herein, numerous alternatives and equivalents will be seen to exist which incorporate the disclosed invention. As a result, the invention is not to be limited by the foregoing embodiments, but only by the following claims.

1. An apparatus for screening a compound by monitoring the interactions of said compound with a specimen having fluorophore loaded target cells, said apparatus comprising:
an optical illumination unit comprising a light source containing wavelengths of light that can stimulate said fluorophore loaded specimen, wherein light from said light source is directed to illuminate said specimen;
a fluorescence separation unit coupled to receive emitted light from said specimen and separate at least three emitted wavelengths of light from said emitted light; and
a fluorescence detection unit coupled to said fluorescence separation unit to count photons emitted by said at least three wavelengths of emitted light.

2. The apparatus of claim 1 wherein said optical illumination unit comprises a laser beam light source.

3. The apparatus of claim 2 wherein said optical illumination unit further comprises a light processing unit coupled to said laser beam light source, said light processing circuit altering the qualities of a light beam from said laser beam light source.

4. The apparatus of claim 3 wherein said light processing unit comprises a filter.

5. The apparatus of claim 3 wherein said light processing unit comprises a beam expander.

6. The apparatus of claim 1 further comprising at least two dichroic mirrors coupled to said optical illumination unit.

7. The apparatus of claim 6 wherein a first dichroic mirror of said at least two dichroic mirrors is coupled to pass light from a first light source and deflects light from a second light source s so that light from said first light source and said second light source merge.

8. The apparatus of claim 7 wherein the last dichroic mirror of said at least two dichroic mirrors deflects light from the previous dichroic mirror and passes emitted light from said specimen.

9. The apparatus of claim 1 wherein said fluorescence separation unit comprises at least two dichroic mirrors.

10. The apparatus of claim 9 wherein said fluorescence separation unit comprises a first dichroic mirror that deflects a first wavelength of light from said light emitted from said specimen.

11. The apparatus of claim 10 wherein said fluorescence separation unit comprises a second dichroic mirror that deflects a second wavelength of light from said light emitted from said specimen and passes a third wavelength of light from light emitted from said specimen.

12. The apparatus of claim 1 wherein said fluorescence separation unit further comprises at least three dichroic polarizer-analyzers and at least three band-limited interference filters.

13. The apparatus of claim 1 further comprising at least three photo-detectors coupled to receive said at least three wavelengths of emitted light.

14. An apparatus for screening a compound by monitoring the interactions of said compound with a specimen having fluorophore-loaded target cells, said apparatus comprising:
an optical illumination unit comprising a light source which generates polarized light;
a plurality of filters coupled to said optical illumination unit to illuminate said specimen;
a fluorescence separation unit comprising at least two filters to direct and separate at least three emitted wavelengths of light from light emitted from said specimen and couple each wavelength of light of said at least three emitted wavelengths of light to a separate dichroic polarizer-analyzer; and
a fluorescence detection unit comprising at least three detectors, each of said detectors comprising a photo-detector.

15. The apparatus of claim 14 wherein said optical illumination unit comprises a laser beam light source.

16. The apparatus of claim 15 further comprising a light processing unit coupled to said laser beam light source, said light processing circuit altering the qualities of a light beam from said laser beam light source.

17. The apparatus of claim 16 wherein said light processing unit comprises a filter and a beam expander.

18. The apparatus of claim 14 wherein said plurality of filters comprises at least two dichroic mirrors to direct light from said laser beam light source and a second light source to said specimen.

19. The apparatus of claim 14 further comprising an inverted microscope coupled to receive light emitted from said specimen.

20. The apparatus of claim 14 wherein said at least two filters of said fluorescence separation unit comprises at least two dichroic mirrors.

21. The apparatus of claim 14 wherein each separate dichroic polarizer-analyzer is coupled to a separate relay lens and interference filter.

22. The apparatus of claim 14 further comprising a computer coupled to said fluorescence detection unit.

23. The apparatus of claim 22 wherein said computer comprises a counter.

24. An apparatus for screening a compound by monitoring its interactions with a specimen having fluorophore-loaded target cells, said apparatus comprising:
an optical illumination unit comprising a light source containing wavelengths that can stimulate said fluorophore-loaded specimen;
a first dichroic mirror that deflects a first wavelength of light from said light emitted from said specimen;
a second dichroic mirror that deflects a second wavelength of light from said light emitted from said specimen and passes a third wavelength of light from said specimen;
at least three dichroic polarizer-analyzers and at least three band-limited interference filters; and
at least three photo-detectors coupled to receive outputs associated with said first, second and third wavelengths of light.

25. The apparatus of claim 24 wherein said optical illumination unit comprises
a first light source;
a second light source;
a third dichroic mirror coupled to receive light from said first light source and said second light source;
a fourth dichroic mirror coupled to receive light from said first light source which is passed by said third dichroic mirror and coupled to receive light from said fourth light source which is deflected by said third dichroic mirror, said fourth dichroic mirror being coupled to deflect said light from said first light source and said second light source to said specimen and pass light emitted from said specimen;

26. The apparatus of claim 25 further comprising a light processing unit.

27. The apparatus of claim 26 wherein said light processing unit comprises a filter.

28. The apparatus of claim 26 wherein said light unit comprises a beam expander.

29. The apparatus of claim 24 further comprising an inverted microscope coupled to receive light emitted from said specimen.

30. The apparatus of claim 24 further comprising a computer coupled to receive outputs of said at least three photo-detectors.

31. An apparatus for screening a compound by monitoring its interactions with a specimen having fluorophore loaded target cells developing a profile of target cells in a specimen, said apparatus comprising:
an argon-ion laser;
a xenon light source;
a first dichroic mirror coupled to receive light from said argon-ion laser and said xenon light source;
a second dichroic mirror coupled to receive light from said argon-ion laser which is passed by said first dichroic mirror and coupled to receive light from said xenon light source which is deflected by said first dichroic mirror, said second dichroic mirror being coupled to deflect said light from said argon-ion laser and said xenon light source to said specimen and pass light emitted from said specimen;
a third dichroic mirror that deflects a first wavelength of light from said light emitted from said specimen;
a fourth dichroic mirror that deflects a second wavelength of light from said light emitted from said specimen and passes a third wavelength of light from said specimen;
at least three dichroic polarizer-analyzers, at least three band-limited interference filters for their respective emission wavelengths;
at least three photo-detectors coupled to receive the outputs associated with said first, second and third wavelengths of light; and
a computer coupled to receive outputs of said at least three photo-detectors.

32. A method of screening a compound by monitoring the interactions of said compound with a specimen having fluorophore loaded target cells, said method comprising the steps of:
coupling a light source to said specimen to illuminate said specimen;
separating at least three wavelengths of light emitted from said specimen, and detecting photons from said three emitted wavelengths of light.

33. The method of claim 32 wherein said step of coupling said light source comprises coupling a laser beam light source to said specimen and further comprises coupling a second light source to said specimen.

34. The method of claim 33 further comprising a step of filtering said light from said laser beam light source.

35. The method of claim 34 further comprising a step of expanding said light from said laser beam light source.

36. The method of claim 33 further comprising a step of focusing light from said laser beamlight source and said second light source on said specimen.

37. The method of claim 33 further comprising a step of filtering said first, second and third wavelengths of light emitted from said specimen.

38. The method of claim 33 further comprising a step of generating a count of photons from said first, second and third wavelengths of light.

39. The method of claim 33 further comprising a step of generating a response profile of said target cells.

40. A method of screening a compound by monitoring the interactions of said compound with a specimen having fluorophore loaded target cells, said method comprising the steps of:
coupling an argon-ion laser to said specimen to illuminate said specimen;
coupling a xenon light source to said specimen to co-axially illuminate said specimen;
separating at least three wavelengths of light emitted from said fluorophore-loaded specimen, detecting photons from said three emitted wavelengths of light;
generating a count of photons from said first, second and third wavelengths of light; and
generating a response profile of said target cells.

41. A method for identifying a pharmaceutically active compound, said method comprising the steps of:
interacting a compound with a specimen containing at least three chemicals of interest;
simultaneously detecting the activities of said at least three chemicals from optical signals emitted from the specimen.

42. The method of claim 41, wherein the said three chemicals comprise a cation indicator, an anion indicator, and a membrane potential indicator.

43. The method of claim 41, wherein the said three chemicals delineate activation of signal transduction pathways and their interactions.

44. The method of claim 42, wherein the said cation comprises Na⁺, K⁺, Ca⁺⁺.

45. The method of claim 42, wherein the said anion comprises Cl⁻, HCO₃⁻.
